(19) United States
(12) Patent Application Publication
(10) Pub. No.: US 2017/0197071 A1
(43) Pub. Date: Jul. 13, 2017

(54) SYSTEM FOR THE ADMINISTRATION OF AN ORAL CARE COMPOSITION

(71) Applicant: KONINKLIJKE PHILIPS N.V., EINDHOVEN (NL)

(72) Inventor: BART GOTTENBOS, BUDEL (NL)

(21) Appl. No.: 15/324,846

(22) PCT Filed: Jun. 29, 2015
(86) PCT No.: PCT/EP2015/064748

§ 371 Date: Jan. 9, 2017

(30) Foreign Application Priority Data
Jul. 11, 2014 (EP) 14176802.8

(51) Int. Cl. A61M 37/00; A61C 17/16; A61N 2/00; A61K 8/04; A61K 8/19; A61Q 11/00; A61K 8/73; A61C 15/04; A61C 17/02

(52) U.S. Cl. CPC A61M 37/00; A61C 15/047; A61C 17/16; A61N 2/004; A61C 17/02; A61K 8/19; A61Q 11/00; A61K 8/736; A61K 8/042; A61M 2037/0007; A61M 2205/0272; A61K 2800/47

(57) ABSTRACT

This is a preliminary amendment. The claims are amended for non-statutory reasons: to remove multiple dependencies, to correct one or more informalities, to remove figure label number(s), and/or to replace European-style claim phraseology with American-style claim language. The scope of the claims have not been changed and no new matter is added.

The amendment to the claims does not address issues of patentability. Applicant(s) reserve(s) the right to continue prosecution of any subject matter canceled, or not claimed because of eliminating multiple dependencies, in this, a divisional, or other continuing application.

Examination of the application is now respectfully requested. If any points remain in issue that may best be resolved through a personal or telephonic interview, the Examiner is respectfully requested to contact the undersigned at the telephone number listed below.

### FIELD OF THE INVENTION

The invention is in the field of oral care, and pertains to a system for the administration of oral care active agents. Particularly, the invention pertains to a gel comprising anti-plaque agents.

### BACKGROUND OF THE INVENTION

The human oral cavity, notably teeth and gums, is generally in need of oral care active agents. Think of, e.g., antiplaque agents, anti-tartar agents, anti-gingivitis agents, anti-bacterial agents, and others.

Such agents are generally administered from toothpastes and/or oral rinse liquids. Due to the typical environment of the oral cavity, e.g. having saliva present, a standard difficulty in the art is that active agents from toothpastes and oral rinses are quickly reducing in concentration after their application. Therefore they cannot protect the mouth for long times, and they need therefore to be applied several times daily.

WO 2008/135957 discloses a method for cleaning dental plaque biofilm from teeth wherein a liquid gelable composition is applied to the teeth. From the composition a gel layer is produced, wherein the gel layer adheres more strongly to the dental plaque bio film than the bio film adheres to the teeth. Ultimately the gel layer is removed from the teeth, and the dental plaque biofilm along therewith, as the dental plaque biofilm adheres to the gel layer.

The in situ formation of the gel can be realized by first administering a gelable liquid (e.g. based on chitosan), and then administering a second liquid (e.g. a sodium hydroxide solution) to bring about gelation. Other methods are disclosed in WO 2008/135957 as well, all involving the in situ formation of the gel. This has certain drawbacks in that not every user will be capable of correctly conducting the gel formation. It would therefore be desirable to provide a gel composition that is ready to use. This, however, brings about another challenge.

It will be interesting to provide oral care active agents in gel particles, particularly with sustained release of active agents therefrom. This would be an attractive solution to increase the concentration of oral care active agents for longer times. Gel particles generally have a low volume of solids (typically 1-2%) and therefore can contain a large volume of active formulation.

However, the actual delivery of active agents from particles administered into the oral cavity, is very challenging, due to the short period of time that such particles will stay in the mouth. E.g., an oral rinse is kept in the mouth for only 30 seconds. Rinsing therefore cannot bring sufficient amounts of particles in contact with the oral surfaces, and most of the particles will be spit out again after application. Also applications with jets will not solve this easily, as in typical fluid flows the mass transport of particles to the surface will be too slow, and the flow too fast. The particles will therefore not adhere, and end up spitted out or swallowed. Moreover, particle adhesion from liquid flow will never result in a full coverage, since adhering particles block a certain “shadow” area around them where new particles cannot adhere. In typical flow cell adhesion tests around 30% particle coverage can be achieved when adhering for several hours.

A similar challenge is encountered when administering active agents via toothpaste. Although the residence time of toothpaste may be longer, as brushing teeth typically occurs for two minutes, the act of brushing and the nature of toothpaste brings about a relatively high amount of low viscous fluid in the mouth, and active agents will be easily swallowed or spitted out.

It is desired to provide a way to administer oral care active agents in such a way that they are less prone to being spitted out or swallowed, and particularly are provided with a longer residence time in the oral cavity, than would be naturally given.

### SUMMARY OF THE INVENTION

In order to better address the foregoing desires, the invention, in one aspect, concerns a system for providing an oral care composition, the system comprising a container holding a ferromagnetic oral care composition, particularly for non-endodontic oral care, and a magnet capable of attracting the ferromagnetic oral care composition.

In another aspect, the invention presents an oral care composition suitable for use in the above system, the composition comprising an oral care active agent, adhesive gel particles, and a ferromagnetic material.

In a further aspect, the invention provides a method of administering a ferromagnetic oral care composition, the method comprising introducing the composition into the oral cavity of a subject, and directing a magnet to a desired position of the oral cavity, so as to attract the ferromagnetic oral care composition.

### BRIEF DESCRIPTION OF THE DRAWINGS

FIG. 1 is macroscope photograph resulting from a test for particle deposition in the interproximal space. without magnet;

FIG. 2 is macroscope photograph resulting from a similar test as in FIG. 1, with a magnet;

FIG. 3 shows optical density, as a measure for bacterial growth, in a comparative test of some embodiments according to the invention and not according to the invention.

### DETAILED DESCRIPTION OF EMBODIMENTS

In a general sense, the invention is based on the judicious insight that the transport of an oral care composition to desired surfaces within the oral cavity, can be highly accelerated using magnetic attraction. To this end, the system of the invention comprises, as its main components, a ferromagnetic oral care composition, and a magnet.

The term “oral care” as used herein refers to non-endodontic applications and treatments. This is opposed to endodontic treatments, such as root canal filling, conducted by a dentist, or surgical treatments conducted by a dental surgeon. Accordingly, the term “oral care agent” or “oral care composition” as used hereinbefore or hereinafter relates to such agents and compositions as are used in non-endodontic oral care. Particularly, this refers to care (particular oral hygiene) applied to surfaces in the oral cavity, such as teeth and gums, i.e. oral surface care and agents and compositions for such oral surface care. Typically, agents and compositions for oral care are administered by end-users themselves. This more particularly refers to toothpastes and oral rinses.

The system of the invention preferably further comprises a dental appliance for cleaning teeth, selected from the group consisting of electric toothbrushes, electric flossing devices, and combinations thereof. Such dental appliances can be provided for various functions. E.g., a typical dental appliance for use with the present invention, is a device for the application of the ferromagnetic oral care composition to the desired surfaces, particularly dental surfaces, in the oral cavity. This typically refers to a toothbrush, preferably an electrical toothbrush, more preferably a sonic power toothbrush having a vibrating brushhead.

An electric flossing device, as is possibly comprised in the system of the invention, refers to such devices that serve to clean the interdental spaces generally by spraying air, by spraying liquid, or a combination thereof. In the system of the invention, such a device can also be of use in order to direct the magnetic oral care composition to the backside of teeth, by the force of spraying, after which the composition can be attracted the to the back surface of the teeth by the action of the magnet.

A typical oral care composition for use in the system of the invention will comprise a gel as a carrier, and one or more oral care active agents. The composition can be directed to a specific use, such as a dedicated antibacterial composition, a dedicated whitening composition, or it can comprise a combination of active agents, such as present, e.g., in toothpaste or mouthwash. Particularly, the composition for use in the system of the invention can be considered as a visco-elastic mouthwash particle, suspended in a regular mouthwash.

It is to be understood that the system can comprise its various parts as separate components, not packaged or provided together.

Particularly, the container holding the ferromagnetic oral care composition can well be provided as a separate entity, e.g., in the form of a bottle or tube holding the composition (comparable to a bottle of mouthwash or a toothpaste tube). Typically, the end-user will finish a plurality of such containers during the life of a magnet. The container can also be attached to the delivery device, particularly as a cartridge adapted for such an attachment, e.g. to an electric toothbrush, flossing device or an oral irrigator, such as a Philips Sonicare AirFloss or Philips Sonicare Toothbrush, with a delivery pump and a magnet.

However, all various configurations are conceivable. E.g., each container holding the ferromagnetic oral care composition can be provided with a magnet, or a package comprising a plurality of such containers, can be provided with a magnet. Typically, in these cases the container and the dental appliance are provided separately.

In an interesting embodiment, the magnet and the dental appliance are provided together. In an interesting embodiment, the magnet is comprised in a dental appliance. This can be seen, e.g., as follows. A toothbrush (either a conventional manual toothbrush or an electrical toothbrush as identified above), is provided with its normal brush functionality (brushhead) on one end. The same appliance can be provided with a magnet, e.g. on the other side of the same end, i.e. opposite the brush, or further removed from the brushhead, such as midway the appliance, or on the other end thereof.

After application of the oral care composition, the magnet can be applied outside of the oral cavity to attract the ferromagnetic oral care composition. The magnet can also be applied within the oral cavity, e.g. under the tongue, in front of a tooth, or behind a tooth, to whichever position it is desired to attract the ferromagnetic composition.

In an interesting further embodiment, the system according to the invention comprises a power module and one or more dental appliance heads that can be removably attached to said power module, wherein the magnet is comprised in a dental appliance head. This typically refers to having an electric toothbrush and/or an electric flossing device, both preferably provided as functional modules in the form of dental appliance heads, and having the magnet as a separate dental appliance head. However, the magnet can also be provided on any dental appliance head also having a different function, such as a brush-head. Typically, the system according to the invention, comprises a magnet, in addition to either or both of an electric toothbrush, and an electric flossing device, as separate dental appliance heads. An interesting system has both an electric toothbrush and an electric flossing device in addition to the magnet. Other dental appliances are conceivable in lieu of, or in addition to, the foregoing devices. E.g., it can be desired to apply the ferromagnetic oral care composition by gentle spreading, rather than brushing or spraying. In fact, the ferromagnetic oral care composition, e.g. in the form of gel particles, can be applied in numerous ways, including but not limited to application by fluid jets (e.g., a slow jet from a syringe), gentle spreading (which can also be referred to as a paint on technique), immersing teeth in a mouthguard type of device, but also simply moving the composition, particularly as a particle fluid, through the mouth.

It will be understood that the function of the magnet is to bring about the attraction to the appropriate dental or periodontal surfaces of one or more oral care active agents. To this end, the ferromagnetic composition is made so as to allow oral care active agents to be attracted by the magnetic fore, and not just to pull ferromagnetic material out of the composition, with the desired oral care active agent staying behind.

To this end, the oral care composition as such is rendered ferromagnetic. To this end, the ferromagnetic oral care composition is preferably in a form selected from the group consisting of solid particles, gels, and combinations thereof. This serves to better secure that attraction by magnetic force will attract the oral care composition, rather than only drawing ferromagnetic material out of such a composition. Preferably, the particles are in the form of a gel, as the cohesive forces within a gel will further aid in attracting the oral care composition as whole.

The composition of the invention can be made by adding ferromagnetic material to a polymeric composition that is capable of forming solid particles. Suitable polymers include polymethyl methacrylate beads, or methyl methacrylate copolymer beads. These are known, e.g., from the field of antibiotics, particularly as used in the treatment of hip infections. Preferred polymeric compositions are gels made of polysaccharide, particularly chitosan. Chitosan is known to be mucoadhesive. This is an advantage for the invention, as a mucoadhesive magnetic gel will not only exert the desired local action as a result of the magnetic material being attracted by the action of a magnet, it will also exert its action for a more prolonged time by being adhesively retained on the spot where it had been attracted to by magnetic force.

Suitable mucoadhesive gels are described, e.g., in Fini et al., Pharmaceutics 2011, 3, 665-679. Other adhesive composition can also be used. Reference is made, e.g., to US 2007/258916, wherein dental compositions are described that are given a high viscosity for better adherence to teeth. Also, a phosphoric acid gel carrier is described that serves to further improve retention on dentin surfaces. It will be appreciated that the skilled person is well aware of various different oral care compositions designed to deliver active agents locally to the teeth, and which will benefit from the presence of magnetic material in accordance with the invention.

The ferromagnetic material preferably is selected from the group consisting of iron, ferric oxide (Fe₂O₃), magnetite (Fe₃O₄), and mixtures thereof. The amount of magnetic material is not particularly critical. The skilled person will be able to establish, for a given composition, an optimal amount that is high enough to be sufficiently divided over the composition, and which is not so high as to too strongly reduce the possible content of the desired oral care active substance present in the composition. Accordingly, the ferromagnetic material is generally present in the composition in an amount of 0.1 wt. % to 10 wt. %, preferably 0.5 wt. % to 5 wt. %, and more preferably 1 wt. %-2 wt. %.

With a view to application in the dental field, white colorants are preferably added (e.g. titanium dioxide), in order to overcome a dark stain as may result from using iron oxides as a magnetic material.

In the system of the invention, the ferromagnetic oral care composition is complemented by the presence of a magnet capable of attracting the ferromagnetic composition. This can be a stand-alone magnet, which the user will be able to put into his or her mouth after administration of the composition. Preferably, however, the magnet is comprised in a dental implement such as a toothbrush, a floss device, or an oral irrigator, such as an AirFloss device. More preferably, these devices are electronic. It will be understood that for the magnet to be capable of attracting the oral care composition, the magnet is adapted to attract rather than repel the ferromagnetic particles.

In an interesting embodiment, the system of the invention comprises a power module and one or more dental appliance heads that can be removably attached to said power module, wherein the magnet is comprised in a dental appliance head. Accordingly, an interesting system comprises a magnet, an electric toothbrush, and an electric flossing device, as separate dental appliance heads.

The system of the invention, as described above, comprises a container holding a ferromagnetic oral care composition. In another aspect, the invention is directed to a ferromagnetic oral care composition as such. Hitherto ferromagnetic oral care compositions have not been known, and the compositions of the invention open up the possibility to achieve localized administration of oral care active agents by magnetic force.

Moreover, the provision of ferromagnetic oral care compositions enhances the possibility of providing oral care active agents in or on solid particles. Without the ferromagnetic nature of such particles, they would be prone to being swallowed or spittted out as explained hereinbefore. By being ferromagnetic, the compositions allow being retained in the oral cavity, and moreover in desired spots thereof, by the attraction of magnetic force.

A further benefit of the invention is that the mass transport of gel particles, or other oral care particles, is not limited by fluid dynamic constraint. As a result, even multiple layers of oral care active particles can be deposited by making use of the invention.

The oral care composition of the invention preferably comprises an oral care active agent, adhesive gel particles, and a ferromagnetic material. The ferromagnetic material is preferably selected from the group consisting of iron, ferric oxide (Fe₂O₃), magnetite (Fe₃O₄), and mixtures thereof. The gel particles preferably are made of chitosan. The composition comprises an oral care active agent preferably selected from the group consisting of antiplaque agents, anti-tartar agents, anti-gingivitis agents, anti-bacterial agents, anti-caries agents, and combinations thereof.

The invention, in a further aspect, also pertains to a method of administering a ferromagnetic oral care composition. The method comprises introducing the composition into the oral cavity of a subject, and directing a magnet to a desired position of the oral cavity, so as to attract the ferromagnetic oral care composition.

In the various aspects of the invention, the design of the delivery device, if any, and the magnet can be easily adapted, dependent on the location where the particles are preferably delivered. E.g., particles can be delivered in the inside of the cheek, by applying the magnet from the outside of the mouth on the cheek. Particles can also be delivered on the tongue by applying a magnet under the tongue.

While the invention has been illustrated and described in detail in the drawings and foregoing description, such illustration and description are to be considered illustrative or exemplary and not restrictive; the invention is not limited to the disclosed embodiments.

For example, it is possible to operate the invention in an embodiment wherein ferromagnetic particles are provided separately from an oral care composition, and are mixed into the oral care composition by an end-user.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain features of the invention are recited in mutually different dependent claims does not indicate that a combination of these features cannot be used to advantage. Any reference signs in the claims should not be construed as limiting the scope.

In sum, we hereby disclose a new method of applying oral care active compositions into the oral cavity. The compositions are ferromagnetic, and can thereby be attracted using the force of a magnet. The invention provides a system comprising a ferromagnetic oral care composition and a magnet capable of attracting the ferromagnetic oral care composition. The components of the system can well be provided separately.

The invention will be further explained hereinafter with reference to the examples and figures. These illustrate the invention, but do not limit it.

### Example 1

Magnetic chitosan gel particles were made by adding 0.5 to 1 m/v% Fe₂O₃ powder to a 2 m/v% chitosan solution in acidic water and gelling the solution in 0.1 M NaOH. The gel was ground to smaller particles (20-200 micron) using an UltraTurrax blender. Different types of particles were made: with or without sodium fluoride and with white colorants (1.5% titanium dioxide).

### Example 2

Interproximal delivery of particles was tested in an anatomical model of the interproximal space made of black Nylon, without and with a magnet applied. In the latter case, a magnet was placed on the occlusal surface. Using a syringe 0.2 ml of particle suspension was brought in the space in 1 s. In the magnet group during the suspension injection a permanent magnet was placed on the occlusal surface, pointing towards the interproximal space to attract the particles to the approximal surface where the magnet was located.

This resulted in a very good deposition when in is a 0.2 ml volume of 1% white magnetic chitosan particles was dispersed into the interproximal space. When the magnet was not present, only few particles adhered, while the magnet resulted in large particle coverage.

The test was done using a model of two molars forming an interproximal gap (teeth touching each other), manufactured from laser sintered black polyamide (Nylon PA11black, Materialise, Leuven, Belgium).

The model consisted of five parts: two teeth, two approximal surfaces and a spacer resembling the gum papillae (hard plastic material as well). The model was a copy of a casted clinical first and second molar set in the 4th quadrant (tooth 6 and 7), except a 0.5 mm gap between the teeth was created to let fluid run through freely. The approximal surface of tooth 6 is referred to as the right surface and the approximal surface of tooth 7 is the left surface, resembling the buccal viewpoint.

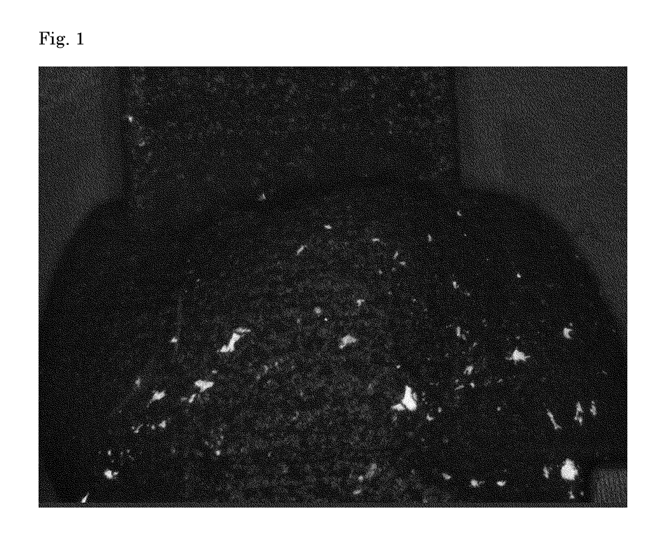

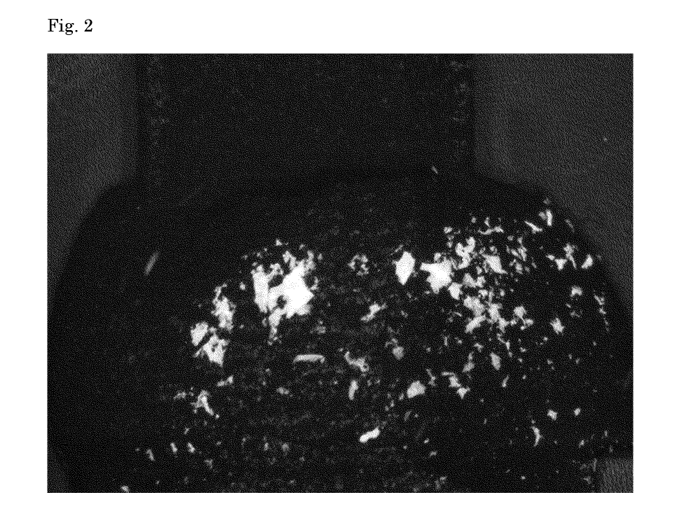

Images were taken with a macroscope (Leica, Z16 APO) and saved as grey scale TIFF with a 16 Bit depth to ensure the high resolution needed for image analysis. Micro Publisher 3.3 enabled footage to be viewed in real time. FIG. 1 shows an image of white ferromagnetic chitosan gel particles on the black approximal model surface after introduction of 0.2 ml 1% particle suspension from the right without a magnet, and FIG. 2 shows the same in the event that a magnet was present at the occlusal surface.

### Example 3

The effect of sodium fluoride loaded chitosan+Fe₂O₃ gel particles was tested on bio film growth. The particles were applied on pre-existing bio films with and without magnet. When a magnet was applied the optical density at the start was strongly increased, showing a larger amount of particles was deposited on the biofilms.

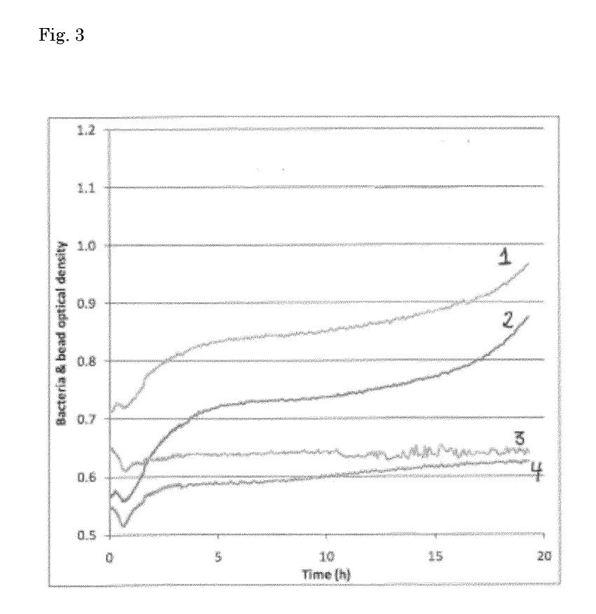

Paramagnetic chitosan+Fe₂O₃ gel particle suspensions, either with or without 13 mM NaF, were placed on 18 h grown Streptococcus mutans biofilms in wells of a 96 well plate. The biofilms were incubated for 15 min, during which in the active magnet groups a magnet was held for 10 s underneath the biofilms. After the incubation the biofilms were washed 3 times with buffer, and subsequently fresh growth medium was added. The plate was incubated at 37° C. in an optical plate reader for 18 h. The optical density was measured during time as a measure of the number of bacteria in the bio film, thus recording bio film growth curves. These curves are presented in FIG. 3. Therein the Y-axis presents the optical density (“bacteria and bead optical density”) as measured. The X-axis represents time in hours (“time (h)”).

In the figure, curves are present representing the results for the four situations tested. The curves are numbered as follows:

(1) magnetic chitosan beads;

(2) chitosan beads;

(3) magnetic NaF chitosan beads;

(4) NaF chitosan beads.

Bio film growth was clearly seen as an optical density increase in the particles not loaded with NaF (curves 1 and 2). When loaded with NaF growth was slowed down (curves 3 and 4), but only when magnetically attracted to the bio film surface the bio film growth was completely stopped (curve 3).

It will be understood that the optical density for the magnetic chitosan beads (curve 1) is higher than that of the chitosan beads without magnetic particles (curve 2), indicating that a higher number of beads is attracted (the optical density being affected by the beads). Also in the event of NaF-containing particles, the optical density of the magnetic beads (curve 3) is higher, as above, as a result of a higher concentration of beads, than in the event of the non-magnetic beads (curve 4). From FIG. 3 it can be seen that the curve of the non-magnetic NaF-containing beads (curve 4) shows an increase in optical density in time, which is thus indicative of bacterial growth. Finally, however, the curve for the magnetic NaF-containing chitosan beads (curve 3) shows no increase in optical density. This shows that the magnetic attraction of the beads towards the bio film actually serves to present a greater anti-bacterial effect than in the event that the beads are not magnetically attracted.

1. A system for providing an oral care composition, the system comprising a container holding a ferromagnetic oral care composition and a magnet capable of attracting the ferromagnetic oral care composition.

2. A system according to claim 1, wherein the oral care composition is a toothpaste or an oral rinse.

3. A system according to claim 1, further comprising a dental appliance for cleaning teeth selected from the group consisting of electric toothbrushes, electric flossing devices, oral irrigators, and combinations thereof.

4. A system according to claim 1 wherein the magnet is comprised in a dental appliance.

5. A system according to claim 1 comprising a power module and one or more dental appliance heads that can be removably attached to said power module, wherein the magnet is comprised in a dental appliance head.

6. A system according to claim 5, comprising a magnet, an electric toothbrush, and an electric flossing device, as separate dental appliance heads.

7. A system according to claim 1 wherein the ferromagnetic oral care composition comprises ferromagnetic particles, preferably selected from the group consisting of iron, ferric oxide (Fe₂O₃), magnetite (Fe₃O₄), and mixtures thereof.

8. A system according to claim 1 wherein the ferromagnetic oral care composition is a gel.

9. A system according to claim 8, wherein the gel comprises gelled chitosan.

10. A system according to claim 1 wherein the magnetic oral care composition is selected from the group consisting of dentifrices, antiplaque compositions, anti-tartar compositions, anti-gingivitis compositions, anti-caries compositions, anti-bacterial compositions, compositions for periodontal treatment, and combinations thereof.

11. An oral care composition suitable for use in a system according to claim 1 the composition comprising an oral care active agent, adhesive gel particles, and a ferromagnetic material.

12. A composition according to claim 11, wherein the ferromagnetic material is selected from the group consisting of iron, ferric oxide (Fe₂O₃), magnetite (Fe₃O₄), and mixtures thereof.

13. A composition according to claim 11, wherein the gel particles are made of chitosan.

14. A composition according to claim 11 wherein the oral care active agent is selected from the group consisting of antiplaque agents, anti-tartar agents, anti-gingivitis agents, anti-bacterial agents, and combinations thereof.

15. A method of administering a ferromagnetic oral care composition, preferably a composition according to claim 11 the method comprising introducing the composition into the oral cavity of a subject, and directing a magnet to a desired position of the oral cavity, so as to attract the ferromagnetic oral care composition.
